(19) United States
(12) Patent Application Publication
(10) Pub. No.: US 2020/0261507 A1
(43) Pub. Date: Aug. 20, 2020

(54) METHOD FOR AVOIDANCE OF BLUE LIGHT DAMAGE BY USING STEM CELL COMPOSITION

(71) Applicant: GROWGENE BIOTECH INC., Taipei City (TW)

(72) Inventors: PEI-CHUAN CHUANG, TAIPEI CITY (TW); YU-WEI CHU, TAIPEI CITY (TW); I-FU CHEN, TAIPEI CITY (TW)

(21) Appl. No.: 16/280,229

(22) Filed: Feb. 20, 2019

(51) Int. Cl. A61K 35/28; C12N 5/0775; A61P 17/16

(52) U.S. Cl. CPC A61K 35/28; C12N 2501/115; A61P 17/16; C12N 5/0662

(57) ABSTRACT

The present invention relates to a method for avoidance of blue light damage by using a stem cell composition. The stem cell composition comprises 10% (v/v) to 50% (v/v) of the conditioned medium of Wharton's Jelly mesenchymal stem cells. The conditioned medium of Wharton's Jelly mesenchymal stem cells is prepared by culturing Wharton's Jelly mesenchymal stem cells in a medium containing human basic fibroblast growth factors for 2 to 5 days, and collecting the medium for centrifugation and filtration.

### BACKGROUND OF THE INVENTION

### Field of the Invention

The present invention relates to a method for avoidance of blue light damage by using a stem cell composition containing a conditioned medium of Wharton's Jelly mesenchymal stem cells.

### Description of Related Art

Blue light is defined as visible light having a wavelength ranging from 380 nm to 500 nm and the light spectrum of blue light is closest to the light spectrum of ultraviolet light in visible light. Blue light has short wavelength and strong energy, so a long-term exposure to blue light can cause cell damage. In modern society, electronics such as computer, communications and consumer electronics (3C products) emitting high amount of blue light are frequently used by people, so a user who uses 3C products frequently is exposed to blue light for a long time. Long-term exposure to blue light may cause macular degeneration by damaging cone cells. In addition, it is reported that exposure to blue light also causes skin cell damage. Currently, a blue filter is disposed on a screen of a 3C product to reduce blue light emitted from the screen. However, there is few composition or skin care product that can be used to reduce blue light damage to cells.

### SUMMARY OF THE INVENTION

The present invention relates to a method for avoidance of blue light damage by using a stem cell composition containing a conditioned medium of Wharton's Jelly mesenchymal stem cells. It comprises applying an effective amount of the stem cell composition containing 10 (v/v) % to 50 (v/v) % of the conditioned medium of Wharton's Jelly mesenchymal stem cells to the skin of a subject in need thereof so as to avoid blue light damage. The conditioned medium of Wharton's Jelly mesenchymal stem cells is prepared by culturing Wharton's Jelly mesenchymal stem cells in a medium containing human basic fibroblast growth factors for 2 to 5 days, and collecting the medium for centrifugation and filtration.

In an embodiment of the present invention, the stem cell composition comprises 25 (v/v) % to 50 (v/v) % of the conditioned medium of Wharton's Jelly mesenchymal stem cells.

In an embodiment of the present invention, the stem cell composition reduces cell death induced by blue light.

In an embodiment of the present invention, the stem cell composition reduces cell death induced by blue light and oxidative stress.

In an embodiment of the present invention, the stem cell composition reduces production of reactive oxygen species induced by blue light in cells.

Therefore, the stem cell composition of the present invention can reduce blue light damage efficiently.

### BRIEF DESCRIPTION OF THE DRAWINGS

FIG. 1 is a diagram showing that a stem cell composition containing a conditioned medium of Wharton's Jelly mesenchymal stem cells reduces blue light induced cell death.

FIG. 2 is a diagram showing that a stem cell composition containing a conditioned medium of Wharton's Jelly mesenchymal stem cells reduces cell death induced by 33 J/cm² of blue light and oxidative stress.

FIG. 3 is a diagram showing that a stem cell composition containing a conditioned medium of Wharton's Jelly mesenchymal stem cells reduces cell death induced by 66 J/cm² of blue light and oxidative stress.

FIG. 4 is a diagram showing that a stem cell composition containing a conditioned medium of Wharton's Jelly mesenchymal stem cells reduces reactive oxygen species production induced by 33 J/cm² of blue light and oxidative stress.

FIG. 5 is a diagram showing that a stem cell composition containing a conditioned medium of Wharton's Jelly mesenchymal stem cells reduces reactive oxygen species production induced by 66 J/cm² of blue light and oxidative stress.

### DETAILED DESCRIPTION OF THE PREFERRED EMBODIMENT

To provide a thorough understanding, the purpose and advantages of the present invention will be described in detail with reference to the accompany drawings.

The present invention relates to a method for avoidance of blue light damage to cells by using a stem cell composition containing a conditioned medium of Wharton's Jelly mesenchymal stem cells (WJMSC). The stem cell composition comprises 10% (v/v) to 50% (v/v) of the conditioned medium of WJMSC cells. The conditioned medium of WJMSC cells is prepared by culturing WJMSC cells in a medium containing human basic fibroblast growth factors (bFGFs) for 2 to 5 days, and collecting the medium for centrifugation and filtration. The stem cell composition containing the conditioned medium of WJMSC cells reduces cell death induced by blue light or blue light combined with oxidative stress. Furthermore, the stem cell composition also reduces production of reactive oxygen species (ROS) in cells.

Hereinafter, exemplary embodiments of the present invention will be described in detail with reference to the accompanying drawings.

1. Preparation of Conditioned Medium of Wharton's Jelly Mesenchymal Stem Cells

(1) Culturing Method of Wharton's Jelly Mesenchymal Stem Cells

Wharton's Jelly mesenchymal stem cell (WJMSC cells, BCRC H-WJ001) used in the present invention were cultured in α-MEM medium supplemented with 10% of fetal bovine serum (FBS) and 4 ng/mL of human basic fibroblast growth factors (bFGFs) in an incubator with a temperature of 37° C. and 5% carbon dioxide (CO₂). The medium was replaced by fresh medium every 2 to 3 days, and the WJMSC cells were sub-cultured when the cell density reaches to 80% confluent.

(2) Preparation of Conditioned Medium of Wharton's Jelly Mesenchymal Stem Cells

WJMSC cells were seeded onto a 10-cm cell culture dishes at a cell density of 5×10⁴ cells/cm², and incubated in a medium of 10% FBS α-MEM medium supplemented with 4 ng/mL bFGFs. The next day, the medium was removed and the WJMSC cells were washed by 1× phosphate-buffered saline (PBS) for three times. Then, 10 mL of α-MEM medium supplemented with 4 ng/mL bFGFs without FBS was added into the 10-cm culture dish for incubation for 48 hours. After 48 hours, the medium was collected and centrifuged at 4° C., 2000 rpm for 10 minutes, and the supernatant was filtered through a 0.22 μm filter unit and used as the conditioned medium of WJMSC cells (named as WJMSC-CM). The WJMSC-CM was aliquoted and stored at −20° C., and was thawed just before use and not re-frizzed.

2. Avoidance of Blue Light Damage to Cells by the WJMSC-CM

Human foreskin fibroblasts (Hs68 cells, BCRC 603800) were cultured in dishes containing 10% FBS DMEM medium, and the Hs68 cells were sub-cultured when the cell density reaches 80% confluent. The Hs68 cells used in the embodiment are 27^{th} to 31^{th} subgeneration of the Hs68 cells.

The blue light source used in this experiment is VitaLux LED lamp. The VitalLux LED lamp provides light wavelengths ranging from 440 nm to 453 nm and mainly provides light wavelengths of 453 nm. A laser power meter (LaserCheck, Coherent) was used for adjusting blue light irradiation power to about 55 mW/cm².

In addition, a handheld far infrared thermometer (IR Thermoter, Extech 42509) was used for detecting temperature of Hs68 cells. The temperature of Hs68 cells was controlled below 31° C. before and after blue light irradiation.

(1) Inhibition Blue Light-Induced Cell Death by a Stem Cell Composition

Hs68 cells were seeded onto a 12-well culture dish at a density of 1×10⁴ cells/well at day 0 and incubated in an incubator with a temperature of 37° C. and 5% CO₂, and a bottom area of each well in the 12-well culture is 3.8 cm². The Hs68 cells were irradiated by blue light at day 3 and day 4. Before blue light irradiation, the medium was removed and the Hs68 cells were incubated in 1×PBS buffer, and then the Hs68 cells were irradiated by 33 J/cm² of blue light for 10 minutes or irradiated by 66 J/cm² of blue light for 20 minutes. After blue light irradiation, the Hs68 cells were washed by 1×PBS buffer and cultured in 1% FBS DMEM medium. After blue light irradiation at day 4, the Hs68 cells were incubated for 48 hours in serum-free DMEM medium or a mixed medium prepared by mixing the WJMSC-CM with the serum-free DMEM medium. When the WJMSC-CM was mixed with the serum-free DMEM medium at a volume ratio of 1:1, the mixed medium are named as “50% (v/v) WJMSC-CM”; and when the WJMSC-CM was mixed with the serum-free DMEM medium at a volume ratio of 1:3, the mixed medium are named as “25% (v/v) WJMSC-CM”. After 48 hours, a relative cell survival rate was examined by MTT assay.

Experimental protocol of MTT assay is simply described below: removing the cell medium of the cells, washing the cells by 1×PBS buffer twice, incubating the cells in a medium containing 0.5 mg/mL of MTT (3-(4,5-Dimethylthiazol-2-yl)-2,5 diphenyltetrazolium bromide) reagent at 37° C. for 2 hours, removing 85% volume of the medium, adding 300 μL dimethyl sulfoxide (DMSO) into the cells and incubation for 10 minutes, measuring an optical density at a wavelength of 450 nm and analyzing relative cell survival rate.

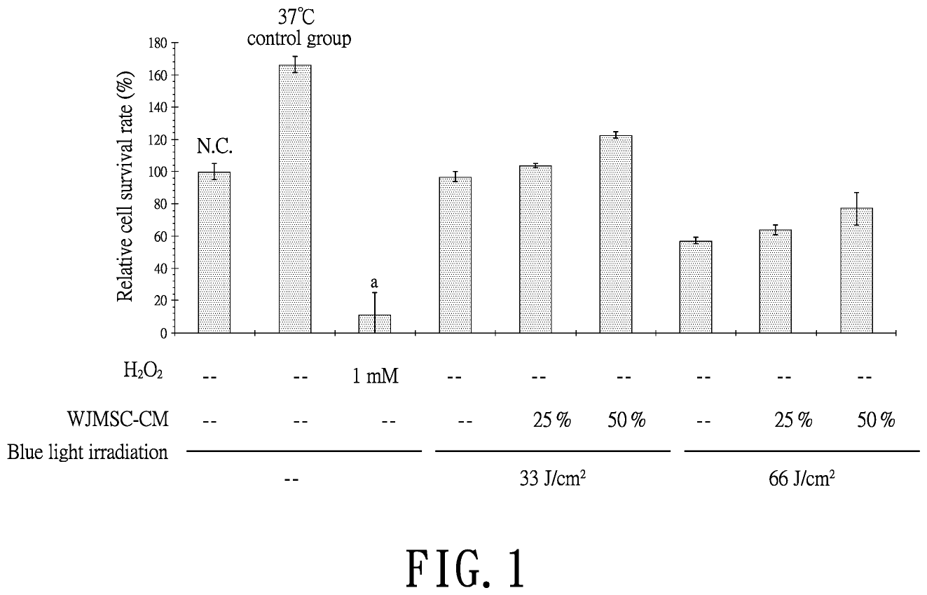

In FIG. 1, Hs68 cells in both negative control (N.C.) group and 37° C. control group are not irradiated by blue light and are incubated at room temperature and 37° C. respectively. The relative cell survival rate of the N.C. group is defined as 100%, and the relative cell survival rate of the 37° C. control group is 170%. A cell death positive control group is also performed by incubating the Hs68 cell in serum-free DMEM medium and treating 1 mM of H₂O₂. Referring to FIG. 1, the relative cell survival rate of the 37° C. control group is 170% compared with the N.C. group. The relative cell survival rate of the cells irradiated by 33 J/cm² of blue light only is not significantly different from the N.C. group. The relative cell survival rate of the cells irradiated by 33 J/cm² of blue light and incubated in 25% (v/v) WJMSC-CM and 50% (v/v) WJMSC-CM are both increased compared with the cells irradiated by 33 J/cm² of blue light only. And the relative cell survival rate of the cells irradiated by 33 J/cm² of blue light and incubated in 25% (v/v) WJMSC-CM and 50% (v/v) WJMSC-CM are different from the N.C. group and the cells irradiated by blue light only significantly (P<0.05). The relative survival rate of the Hs68 cells irradiated by 66 J/cm² of blue light only are decreased significantly compared with the N.C. group and the 37° C. control group (P<0.05) which suggested that blue light indeed causes cell damages. The relative cell survival rate are both increased of the cells irradiated by 66 J/cm² of blue light and incubated in 25% (v/v) WJMSC-CM and 50% (v/v) WJMSC-CM compared with the cell irradiated by 66 J/cm² of blue light only.

(2) Cell Toxicity Assay

Hs68 cells were seeded onto a 96-well culture dish at a density of 2×10⁴ cells/well at day 0 and incubated in an incubator with a temperature of 37° C. and 5% carbon dioxide (CO₂). The Hs68 cells were irradiated by blue light at day 1 and day 2. Before blue light radiation, the medium was removed and the Hs68 cells were incubated in 1×PBS buffer. Then the Hs68 cells were irradiated by 33 J/cm² of blue light for 10 minutes or irradiated by 66 J/cm² of blue light for 20 minutes. After blue light irradiation, the Hs68 cells were washed by 1×PBS buffer and cultured in 1% FBS DMEM medium. After blue light irradiation at day 2, the Hs68 cells were incubated in serum-free DMEM medium or the mixed medium prepared by the WJMSC-CM with the serum-free DMEM medium, and the cells were also treated with DMSO or H₂O₂. The cells were incubated for 24 hours after blue light irradiation at day 2. After 24 hours, the relative cell survival rate was examined by using a CCK-8 (cell counting kit-8) kit (Sigma, cat no. 96992) simply described below: adding 10 μL of CCK-8 reagent into the cells and incubating for 2 hours, and an optical density at a wavelength of 450 nm is examined. In this experiment, treating of H₂O₂ to the cells is used to simulate an oxidative stress situation.

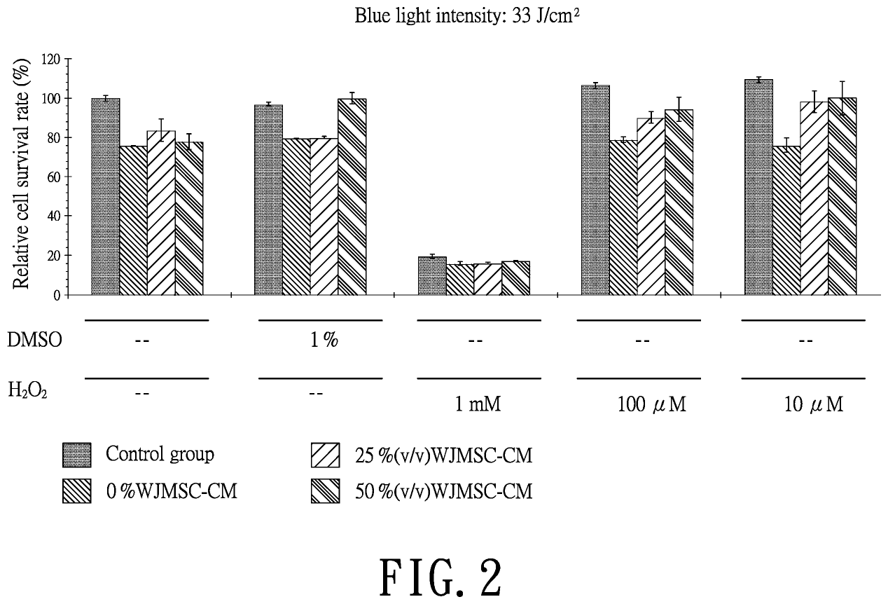

FIG. 2 is a diagram showing relative cell survival rates of cells irradiated by 33 J/cm² of blue light. The cells in the control group are incubated in 10% FBS DMEM medium after blue light irradiation. The cells in the “0% WJMSC-CM” group are incubated in serum-free DMEM medium after blue light irradiation. In the cells irradiated by 33 J/cm² of blue light only in FIG. 2, the relative cell survival rate of the cells incubated in 25% (v/v) WJMSC-CM and 50% (v/v) WJMSC-CM are not significantly different from the cells incubated in 0% (v/v) WJMSC-CM. In the cells irradiated by 33 J/cm² of blue light and incubated with 1% DMSO in FIG. 2, the relative cell survival rate of the cell incubated in 50% (v/v) WJMSC-CM is significantly increased compared with the cells incubated in 0% WJMSC-CM (P<0.05). In the cells irradiated by 33 J/cm² of blue light and incubated with 1 mM H₂O₂ in FIG. 2, the relative cell survival rates of the experimental groups are similar and are all lower than the cell survival rates of the cells irradiated by 33 J/cm² of blue light only. In the cells irradiated by 33 J/cm² of blue light and incubated with 100 μM H₂O₂ in FIG. 2, the cell survival rate of the cells incubated in 25% (v/v) WJMSC-CM and 50% (v/v) WJMSC-CM are all increased significantly compared to the cells incubated in 0% (v/v) WJMSC-CM (P<0.05). In the cells irradiated by 33 J/cm² of blue light and incubated with 10 μM H₂O₂ in FIG. 2, the cell survival rate of the cells incubated in 25% (v/v) WJMSC-CM and 50% (v/v) WJMSC-CM are also increased significantly compared to the cells incubated in 0% (v/v) WJMSC-CM (P<0.05).

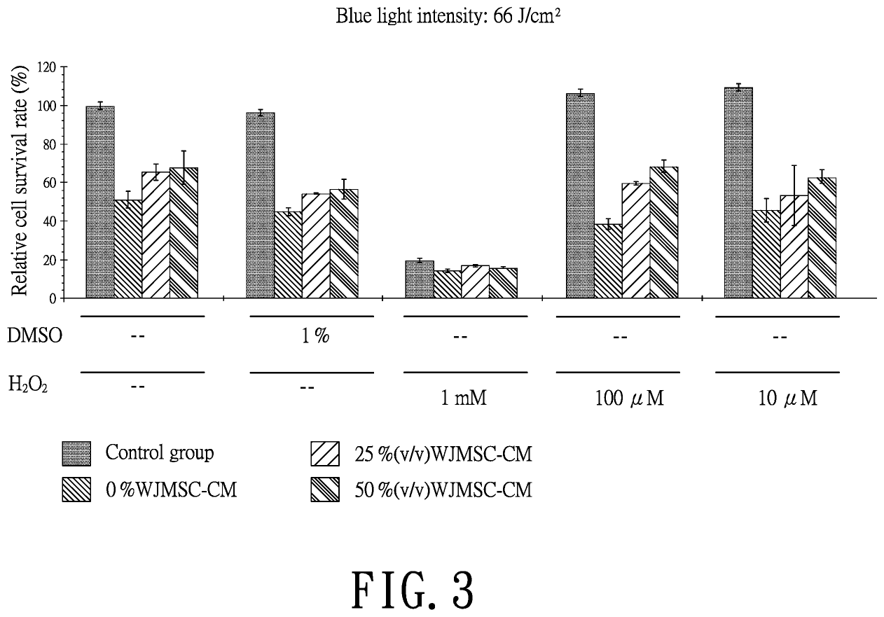

FIG. 3 is a diagram showing relative cell survival rates of cells irradiated by 66 J/cm² of blue light. The cells in the control group are incubated in 10% FBS DMEM medium after blue light irradiation. The cells in “0% (v/v) WJMSC-CM” group are incubated in serum free DMEM medium after blue light irradiation. In the cells irradiated by 66 J/cm² of blue light and incubated without DMSO and H₂O₂ in FIG. 3, the relative cell survival rate of the cells incubated in 25% (v/v) WJMSC-CM and 50% (v/v) WJMSC-CM are increased significantly compared with the cells incubated in 0% (v/v) WJMSC-CM (P<0.05). In the cells irradiated by 66 J/cm² of blue light and incubated with 1% DMSO, the relative cell survival rates of the cells incubated in 25% (v/v) WJMSC-CM and 50% (v/v) WJMSC-CM are significantly increased compared with the cells incubated in 0% (v/v) WJMSC-CM (P<0.05). In the cells irradiated by 66 J/cm² of blue light and incubated with 1 mM H₂O₂ in FIG. 3, the relative cell survival rates of cells incubated in 25% (v/v) WJMSC-CM and 50% (v/v) WJMSC-CM are slightly increased compared with the cells incubated in 0% (v/v) WJMSC-CM and are all lower than the cells irradiated by 66 J/cm² of blue light only. In the cells irradiated by blue light and treated with 100 μM H₂O₂ in FIG. 3, the cell survival rates of the cells incubated in 25% (v/v) WJMSC-CM and 50% (v/v) WJMSC-CM are increased significantly compared with the cells incubated in 0% (v/v) WJMSC-CM (P<0.05). In the cells irradiated by 66 J/cm² of blue light and treated with 10 μM H₂O₂ in FIG. 3, the cell survival rates of the cell incubated in 25% (v/v) WJMSC-CM and 50% (v/v) WJMSC-CM are also increased significantly compared with the cells incubated in 0% WJMSC-CM (P<0.05).

According to FIG. 2 and FIG. 3, the mixed medium containing the WJMSC-CM in the present invention indeed avoids cell damage and cell death induced by blue light and oxidative stress.

(3) Reactive Oxygen Species Production

Hs68 cells were seeded onto a 96-well culture dish at a density of 2×10⁴ cells/well at day 0 and incubated in an incubator with a temperature of 37° C. and 5% CO₂. The Hs68 cells were irradiated by blue light at day 1 and day 2. Before blue light radiation, the medium was removed and the Hs68 cells were incubated in 1×PBS buffer, then the Hs68 cells were irradiated by 33 J/cm² of blue light for 10 minutes or irradiated by 66 J/cm² of blue light for 20 minutes. Before blue light irradiation at day 2, the cells were washed by 1×PBS buffer for few times and incubated in DMEM medium supplemented with 2′,7′-dichlorodihydrofluorescein diacetate (H2DCFDA) reagent and incubated at 37° C. for 30 minutes; then the cells were washed by 1×PBS buffer and incubated in 1×PBS buffer for examining an optical density at a wavelength of 525 nm; and the cells were subjected to blue light irradiation. After blue light irradiation at day 2, the Hs68 cells were incubated in serum-free DMEM medium or the mixed medium prepared by mixing the WJMSC-CM with the serum-free DMEM medium, and the cells were also treated with DMSO or H₂O₂. In this experiment, adding of H₂O₂ to the cells is used to simulate an oxidative stress situation. After incubation for 30 minutes, the optical density at wavelength of 525 nm is re-examined.

H2DCFDA reagent is a cell-permeant indicator for reactive oxygen species (ROS). After entering cells, H2DCFDA reagent is to converted to non-fluorescent DCFH molecule by esterase of the cells which cannot pass through the cell membrane and is retained in the cells. When ROS is generated in the cells, the non-fluorescent DCFH molecule is oxidized by ROS and then converted to fluorescent DCF molecule which has an optical density at a wavelength of 525 nm, so the ROS is content in the cells can be evaluated by measuring optical density at wavelength of 525 nm.

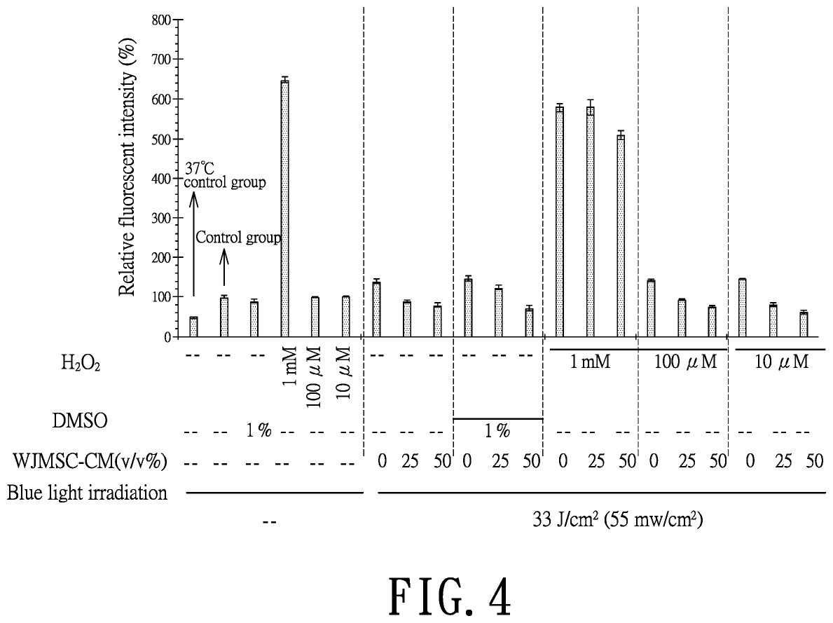

In this experiment, N.C. group is used as a baseline for calculating relative fluorescent intensities of the experimental groups. The cells of N.C. group and 37° C. control group are not irradiated by blue light and are incubated at room temperature and 37° C. respectively. Referring to FIG. 4 and table 1, the ROS contents of the cells without blue light irradiation and treated with or without H₂O₂ are examined. The ROS contents in cells treated with 1 mM H₂O₂ is increased compared with the N.C. group and 37° C. control group. Referring to FIG. 4 and table 2, the cells are irradiated by 33 J/cm² of blue light and treated with 1% DMSO, the ROS contents in the cells incubated in 25% (v/v) WJMSC-CM and 50% (v/v) WJMSC-CM are significant lower than the cells incubated in 0% (v/v) WJMSC-CM (P<0.05). Referring to FIG. 4 and table 3, the cells are irradiated by 33 J/cm² of blue light and treated with H₂O₂, the ROS contents of the cells incubated in 25% (v/v) WJMSC-CM and in 50% (v/v) WJMSC-CM are significant lower than the cells incubated in 0% (v/v) WJMSC-CM (P<0.05).

TABLE 1

| | Not irradiated by blue light | | | | | |
|---|---|---|---|---|---|---|
| | 37° C. | | 1% | 1 mM | 100 μM | 10 μM |
| | control | N.C. | DMSO | H₂O₂ | H₂O₂ | H₂O₂ |
| Relative | 0 | 53 | 43 | 601 | 53 | 55 |
| fluorescent | | | | | | |
| intensity | | | | | | |

TABLE 2

| | Irradiated by 33 J/cm² of blue light | | | | | |
|---|---|---|---|---|---|---|
| | | | | Not treated with H₂O₂, | | |
| | | | | and treated with 1% | | |
| H₂O₂ | Not treated with H₂O₂ | | | DMSO | | |
| WJMSC-CM | 0 | 25 | 50 | 0 | 25 | 50 |
| (% (v/v)) | | | | | | |
| Relative | 92 | 43 | 33 | 99 | 76 | 26 |
| fluorescent | | | | | | |
| intensity | | | | | | |

TABLE 3

| | Irradiated by 33 J/cm² of blue light | | | | | | | | |
|---|---|---|---|---|---|---|---|---|---|
| H₂O₂ | 1 mM | | | 100 μM | | | 10 μM | | |
| WJMSC-CM | 0 | 25 | 50 | 0 | 25 | 50 | 0 | 25 | 50 |
| (% (v/v)) | | | | | | | | | |
| Relative | 532 | 531 | 461 | 96 | 45 | 29 | 99 | 33 | 15 |
| fluorescent | | | | | | | | | |
| intensity | | | | | | | | | |

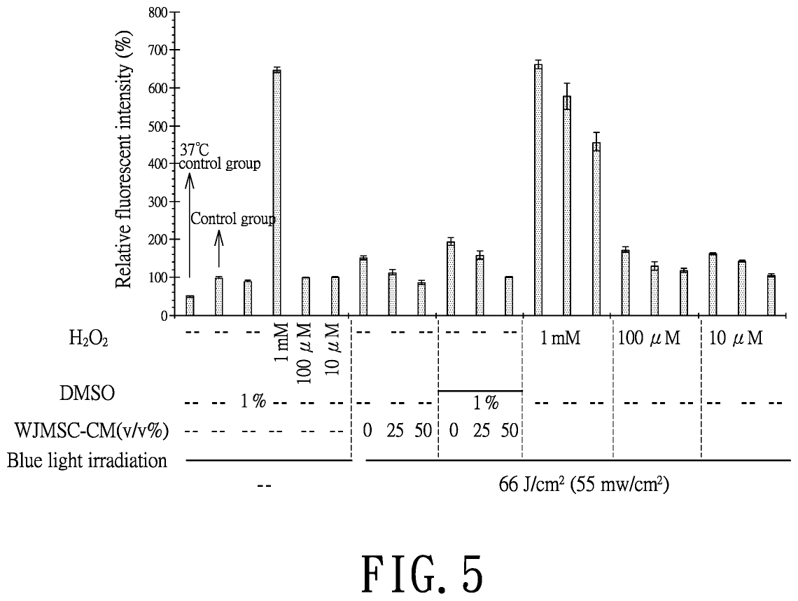

Referring to FIG. 5 and table 1, the ROS contents of the cells without blue light irradiation and treated with or without H₂O₂ are examined, the ROS content in the cell treated with 1 mM H₂O₂ is increased. Referring to FIG. 5 and table 4, the cells are irradiated by 66 J/cm² of blue light and treated with 1% DMSO, the ROS contents in the cells incubated in 25% (v/v) WJMSC-CM and 50% (v/v) WJMSC-CM are significant lower than the cells incubated in 0% (v/v) WJMSC-CM (P<0.05). Referring to FIG. 5 and table 5, the cells are irradiated by 66 J/cm² of blue light and treated with 10 μM to 1 mM H₂O₂, the ROS contents in the cells incubated in 25% (v/v) WJMSC-CM or 50% (v/v) WJMSC-CM are significantly lower than the cells incubated in 0% (v/v) WJMSC-CM (P<0.05).

TABLE 4

| | Irradiated by 66 J/cm² of blue light | | | | | |
|---|---|---|---|---|---|---|
| | | | | Not treated with H₂O₂, | | |
| | | | | and treated with 1% | | |
| H₂O₂ | Not treated with H₂O₂ | | | DMSO | | |
| WJMSC-CM | 0 | 25 | 50 | 0 | 25 | 50 |
| (% (v/v)) | | | | | | |
| Relative | 104 | 66 | 39 | 148 | 111 | 53 |
| fluorescent | | | | | | |
| intensity | | | | | | |

TABLE 5

| | Irradiated by 66 J/cm² of blue light | | | | | | | | |
|---|---|---|---|---|---|---|---|---|---|
| H₂O₂ | 1 mM | | | 100 μM | | | 10 μM | | |
| WJMSC-CM | 0 | 25 | 50 | 0 | 25 | 50 | 0 | 25 | 50 |
| (% (v/v)) | | | | | | | | | |
| Relative | 616 | 532 | 410 | 125 | 82 | 72 | 114 | 95 | 59 |
| fluorescent | | | | | | | | | |
| intensity | | | | | | | | | |

According to the experiments above, blue light with wavelengths ranging from 440 nm to 453 nm indeed damages the cells including inhibiting cell growth, inducing cell death or increasing ROS production in cells. In addition, combination of blue light and oxidative stress also induces cell death and increases ROS production in cells. However, when the cells are incubated in a mixed medium containing at least 10% (v/v) WJMSC-CM, the cell survival rate is increased and the content of ROS is decreased in the cells which indicated that the WJMSC-CM in the present invention avoids blue light damages to cells efficiently.

What is claimed is:

1. A method for avoidance of blue light damage to cells by using a stem cell composition containing a conditioned medium of Wharton's Jelly mesenchymal stem cells, comprising: applying an effective amount of the stem cell composition containing 10 (v/v) % to 50 (v/v) % of the conditioned medium of Wharton's Jelly mesenchymal stem cells to the skin of a subject in need thereof so as to avoid blue light damage, wherein the conditioned medium of Wharton's Jelly mesenchymal stem cells is prepared by culturing Wharton's Jelly mesenchymal stem cells in a medium containing human basic fibroblast growth factors for 2 to 5 days, and collecting the medium for centrifugation and filtration.

2. The method as claimed in claim 1, wherein the stem cell composition contains 25% (v/v) to 50% (v/v) of the conditioned medium of Wharton's Jelly mesenchymal stem cells.

3. The method as claimed in claim 1, wherein the stem cell composition reduces cell death induced by blue light.

4. The method as claimed in claim 1, wherein the stem cell composition reduces cell death induced by blue light and oxidative stress.

5. The method as claimed in claim 1, wherein the stem cell composition reduces production of reactive oxygen species in cells, induced by blue light.
